(19) United States
(12) Plant Patent Application Publication
(10) Pub. No.: US 2002/0059672 P1
(43) Pub. Date: May 16, 2002

(54) Lily plant named 'Soldera'

(76) Inventor: Cees A. van der Voort, JN KATWIJK (NL)

Correspondence Address: Foley & Lardner, Washington Harbour, 3000 K Street NW, Suite 500, Washington, DC, 20007-5109, US

(21) Appl. No.: 09/521,607

(22) Filed: Mar. 9, 2000

(51) Int. Cl. A01H 5/00

(52) U.S. Cl. PLT/315

(57) ABSTRACT

A new and distinct cultivar of Lily plant named ‘Soldera’ characterized by its large erect to vertical flowers, petals with curled edges, red-purple color and vigorous growth.

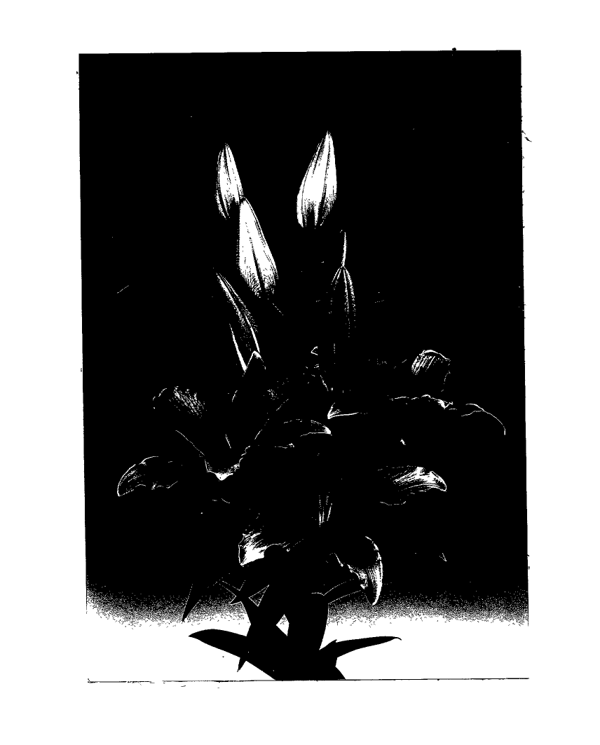

### BACKGROUND OF THE INVENTION

The present invention comprises a new and distinct cultivar of lily plant, botanically known as Lilium L., and hereinafter referred to by the cultivar name ‘Soldera’.

‘Soldera’ is a product of a planned breeding program which had the objective of creating new lily cultivars having many large erected flowers and vigorous growth.

‘Soldera’ was originated from a hybridization made by the inventor in a controlled breeding program in Rijnsburg, the Netherlands in 1986. The male and female parents were unnamed seedlings of Lilium L. oriental hybrids. Both parents are proprietary cultivars used in the breeding program.

‘Soldera’ was discovered and selected as one flowering plant within the progeny of the stated cross by the inventor, Cees A. van der Voort, during the months of May and June 1986 in a controlled environment in Rijnsburg, the Netherlands.

The first act of asexual reproduction of ‘Soldera’ was accomplished when scales were taken from the initial selection in October 1988 in a controlled environment in Rijnsburg, the Netherlands by, or under the supervision of, Cees A. van der Voort. Horticultural examination of selected units initiated in May, 1988 has demonstrated that the combination of characteristics as herein disclosed for ‘Soldera’ are firmly fixed and are retained through successive generations of asexual reproduction.

### BRIEF DESCRIPTION OF THE INVENTION

The following traits have been repeatedly observed and are determined to be basic characteristics of ‘Soldera’ which in combination distinguish this lily as a new and distinct cultivar:

1. many large, erect flowers;

2. slim tepals with curled edges;

3. soft pink color with contrasting orange pollen; and

4. vigorous growth habit.

‘Soldera’ has not been observed under all possible environmental conditions. The phenotype of the new cultivar may vary significantly with variations in environment such as temperature, light intensity, and daylength without any change in genotype. The following observations, measurements, and comparisons describe plants grown in the experimental research station CPRO-DLO, Wageningen, the Netherlands under conditions which approximate those generally used in commercial practice.

Of the many commercial cultivars known to the present inventor, no other cultivars are similar in comparison to ‘Soldera’.

### BRIEF DESCRIPTION OF THE DRAWING

The accompanying color photographic illustration shows typical plant and flower characteristics of ‘Soldera’, with colors being as true as possible with illustrations of this type.

### DETAILED BOTANICAL DESCRIPTION

In the following description, color references are made to The Royal Horticultural Society (RHS) Colour Chart. The color values were determined between 12 pm and 2 pm in June 1997 at the experimental research station CPRO-DLO, Wageningen, the Netherlands.

Origin: Selected crossing from a controlled hybridization program

Parentage:

Female parent.—Unnamed seedling

Male parent.—Unnamed seedling

Classification:

Botanical.—A hybrid of the genus Lilium L.

Commercial.—Oriental Lily cv. ‘Soldera’

Plant:

Form.—A single stem carrying numerous, alternately arranged pedicels

Height.—Short to medium for the mature plant, approximately 85 cm, depending on cultivation conditions

Growth habit.—Vigorous

Stem.—Circular in transverse section; anthocyanin pigmentation weakly present in spots and stripes; internodal length uniform

Foliage:

Quantity.—Approximately 35-45, depending on cultivation conditions

Size of leaf.—Approximately 8½ cm long and 3 cm wide, depending on cultivation conditions

Shape of leaf.—Generally ovate

Texture.—Hardly any pubescence

Color.—Light-green

Bulb.—Size: 16 to 18 cm in circumference Color: White/pink

Bud:

Form.—Tapering like a lancet

Size.—Long, 10 cm in length, depending on cultivation conditions

Opening.—Unfolds normally in standard cultivation conditions

Color.—Red-purple, RHS 60D just before opening

Tepals.—Folded, three visible

Flower:

Blooming habit.—Raceme

Size.—Medium, approximately 16 cm, depending on cultivation conditions

Borne.—Erect to vertical along a longitudinal axis

Shape.—Generally, hexagonal star in form and bowl-shaped in cross-section

Tepalage.—Number of Tepals: 6 Arrangement: Three inner and three outer Color: Upper Surface: Red-purple, RHS 60D, turning bluish as it ages Lower Surface: Red-purple, RHS 60D, turning bluish as it ages

Pedicel.—Length: Medium, depending on cultivation conditions Form: Tapering like a lancet

Texture.—Papillose

Disease resistance.—Medium

Fragrance.—Present

Lasting quality.—Excellent

Reproductive Organs:

Stamens and anthers.—Arrangements: Anthers form a generally circular pattern at their upper ends Number: 6 Length: Medium, depending on cultivation conditions Filaments color: Yellowish-green

Pollen color.—Light-brown

Anther color.—Purplish-red

Pistils.—White/yellow

Stigma.—Dark-purple

Ovaries.—Triangdar in shape

I claim:

1. A new and distinct cultivar of Lily plant named ‘Soldera’, as illustrated and described herein.
